(19) United States
(12) Patent Application Publication
(10) Pub. No.: US 2023/0357125 A1
(43) Pub. Date: Nov. 9, 2023

(54) Process for Catalytic Reduction of Nitro Compounds to Amines

(71) Applicant: AMPAC Fine Chemicals, Rancho Cordova, CA (US)

(72) Inventor: Zuolin Zhu, Sacrament, CA (US)

(21) Appl. No.: 17/738,875

(22) Filed: May 6, 2022

(51) Int. Cl. C07C 209/36; B01J 23/72

(52) U.S. Cl. CPC C07C 209/36; B01J 23/72

(57) ABSTRACT

Disclosed is a process for the catalytic reduction of nitro compounds to their corresponding amine products with bisulfite as the reductant or chemicals that can be converted into bisulfite under reaction condition. This novel process uses non-Noble metal catalysts and affords quantitative conversion for numerous different kinds of nitro compounds under mild reaction conditions.

### TECHNICAL FIELD

The invention relates to economic and green processes for the catalytic reduction of nitro groups to amines.

### BACKGROUND

The reductions of nitro compounds to amines are important chemical transformations in organic synthesis, and in environmental protection. Nitro derivatives are often starting materials in organic synthesis, in which they are generally used as precursors of amino derivatives. Many aromatic nitro derivatives are commercially available or obtainable by nitration of suitable aromatic precursors. Aliphatic nitro derivatives are generally accessible by Michael or Henry reactions. Nitro compounds are also chemically resistive, reactive noxious waste and persistent pollutants in waste streams.

The reduction of nitro derivatives to amines is typically carried out by catalytic hydrogenation and transfer hydrogenation (Chem. Rev. 2019, 119, 2611-2680; Org. Process Res. Dev., 2018, 22, 430-445) or by various other processes, such as sodium borohydride in the presence of a catalyst (J.Chem.Soc.Pak., 2016, 38, 679-684; Catal. Lett. 2008, 123, 264-268), or hydrazine activated with a suitable catalyst (Adv. Synth. Catal. 2007), or with metals such as carbonyl iron powder, iron, zinc or tin (Organic Lett. 2017, 19, 6518-6521; Tetrahedron Lett. 2003, 44, 7783-7787; Organic Lett. 2014, 16, 98-101), reduction with low valent sulfur reagents such as sulfide and dithionite, and also processes that use samarium iodide (J. Org. Chem. 2001, 66, 919-924) and complexes of molybdenum and palladium (Org. Lett. 2005, 7, 5087-5090), to name but a few.

Classically, prior art reductions are conducted under harsh conditions, or generate large amounts of waste, including strong acids and various metals, such as iron, zinc, samarium, or tin (Vogel, A. I. et al. Textbook of Practical Organic Chemistry, 5th ed; J. Org. Chem. 2001, 66, 919-924). Although effective in scaled industrial processes, in certain cases, these reductions present significant safety and environmental issues associated with their use. Several protocols have been reported in recent years to address these concerns. Catalytic hydrogenation using transition metal catalysis has received high interest, but this approach exhibits limited selectivity in the presence of other reducible functional groups (Pandarus, V.; Ciriminna, R.; Beland, F.; Pagliaro, M. Catal. Sci. Technol. 2011, 1, 1616).

Other currently known reduction methods also have certain drawbacks. Catalytic transfer hydrogenation has chemo-selectivity issues; hydrazine is highly and easily combustible, extremely toxic, caustic, probably carcinogenic, and the conversion is very low if simple metal salts is used as the catalyst such as copper sulfate or copper chloride. Specific organometallic complexes such as metal phthalocyanines are needed as the catalyst (Sharma, U.; Kumar, P.; Kumar, N.; Kumar, V.; Singh, B., Adv. Sybth. Catal., 2010, 352, 1834); HSiCl₃ is highly flammable and toxic, diboron compounds are carcinogens or have risk to form carcinogen byproducts (EPA 635/04/052, 2004; Org. Process Res. Dev. 2015, 19, 1507-1516), vasicine and DHA (dihydroanthracene) are very expensive (Org. Process Res. Dev. 2018, 22, 430-445), phosphine ligands always caused isolation issues and have potential pollution, the toxicity of tin salts obviously involves serious problems relating to the wastewater disposal processes and potential pollution of the reaction products.

As a further example, although HSiCl₃ is a strong reductant, it is a highly flammable liquid with boiling point at 31.8° C. and is toxic, reduction of nitro compounds with HSiCl₃ gave low conversion (U.S. Pat. No. 9,284,258B2) for some important intro compounds: such as only 40% for 4-N-acetyl nitrobenzene, and 47% for 4-nitrobenzoic acid. Hydrazine as reductant and copper salts as catalyst, only gave about 10% conversion at 70° C. for 4-nitrobenzonitrile (Sharma, U.; Kumar, P.; Kumar, N.; Kumar, V.; Singh, B., Adv. Synth. Catal., 2010, 352, 1834).

There is consequently a great need to develop new processes of reducing nitro derivatives to amines, and converting high toxic non-biodegradable or very low biodegradable nitro pollutants into readily biodegradable amines, in particular for the development of new sustainable methodologies (U. Sharma, P. K. Verma, N. K. V. Kumar, M. Bala, B. Singh, Chem. Eur. J. 2011, 17, 5903) which are also economical. In this context, it would be advantageous to develop alternative reduction processes, with non-toxic reagents and low environmental impact, especially organic molecules with a plurality of functional groups.

Bisulfite, such as sodium bisulfite, is a non-toxic, very low-cost reagent, and is a weak reductant suitable for reduction of nitro groups as disclosed herein. In addition, the product of bisulfite after reduction is sulfate. Sulfate is an environmental benign ion, and normally is also a good aid for the isolation of organic molecules.

### SUMMARY

An aim of the present disclosure is to provide an economic green process for the reduction to amine of a nitro group present in an aliphatic, aromatic or heteroaromatic compound, in which said compound is reacted with bisulfite in the presence of a suitable catalyst. The process may be considered chemo-selective, as it reduces the nitro groups without reacting with other functional groups present in the molecule, including those which can be attacked, for example, by a hydrogenation process or by strong reductant.

The disclosed processes give quantitative conversion for many kinds of nitro compounds, such as aliphatic and aromatic compounds under mild reaction conditions.

In an embodiment, disclosed is a process for the catalytic reduction of a nitro group-containing compound to an amine product, comprising:
a) dissolving the nitro-group containing compound in solvent;
b) adding a bisulfite compound to the solvent in an amount from about 2.0 to about 8.0 molar equivalents to the nitro group of the nitro group-containing compound;
c) mixing an aqueous solution of a catalyst with the solvent, wherein the volume of the aqueous solution is from about 2% to about 20% of the volume of the solvent, and wherein the catalyst is present in an amount from about 0.05 to about 1 molar equivalents to the nitro group of the nitro group-containing compound; and
d) reacting the nitro group-containing compound with the bisulfite compound in the presence of the catalyst under reaction conditions of temperature in the range from about 20° C. to about 70° C. and atmospheric pressure for a period from about 10 minutes to about 72 hours to obtain the amine product.

In further embodiments, steps, a), b), and c) are performed in any simultaneous or consecutive grouping or ordering.

In further embodiments, the nitro group-containing compound is selected from the group consisting of aliphatic, aromatic and heterocyclic compounds and mixtures thereof.

In further embodiments, the catalyst is a transition metal catalyst containing an ion selected from the group consisting of manganese, iron, cobalt, nickel, and copper, and including salts, complexes, and mixtures thereof.

In further embodiments, the bisulfite compound is selected from the group consisting of sodium bisulfite, potassium bisulfite, ammonium bisulfite, and mixtures thereof.

In further embodiments, the bisulfite compound is sodium bisulfite.

In further embodiments, about 95% of the nitro-group starting material is reduced to an amine product.

In further embodiments, the nitro group of the nitro group-containing compound is selectively reduced by the bisulfite reactions without side reactions with one or more substituents on the nitro group-containing compound.

In further embodiments, the process further comprises adding a buffering agent to the process in an amount of from about 1 to about 10 molar equivalents to the nitro group of the nitro group-containing compound, wherein the buffering agent is a tertiary organic amine selected from triethylamine (TEA), triethanolamine (TEOA), N, N-diethylisopropylamine, N, N-diisopropylethylamine (DIPEA), tripropylamine, trioctylamine or basic inorganic salts selected from sodium acetate, disodium phosphate, sodium sulfite, and sodium hydrosulfite, and mixtures thereof.

In further embodiments, the solvent is dimethylsulfoxide (DMSO).

In further embodiments, the process further comprises the following steps:
e) monitoring the conversion of the nitro-group containing compound to products by liquid or gas chromatography; and
f) adding the buffering agent to the solvent to push the reaction to completion if the nitro-group containing compound is detected in step (d).

In further embodiments, the bisulfite is formed in situ from a bisulfite precursor.

In an embodiment, disclosed is a process for the catalytic reduction of a nitro group-containing compound to an amine product, comprising:
a) dissolving the nitro-group containing compound in solvent at a concentration higher than about 50 mM;
b) adding a bisulfite compound to the solvent in an amount from about 2.0 to about 8.0 molar equivalents to the nitro group of the nitro group-containing compound;
c) mixing an aqueous solution of a catalyst with the solvent, wherein the volume of the aqueous solution is from about 2% to about 20% of the volume of the solvent, and wherein the catalyst is present in an amount from about 0.05 to about 1 molar equivalents to the nitro group of the nitro group-containing compound; and
d) reacting the nitro group-containing compound with the bisulfite compound in the presence of the catalyst under reaction conditions of temperature in the range from about 20° C. to about 70° C. and atmospheric pressure for a period from about 10 minutes to about 72 hours to obtain the amine product.

In an embodiment, disclosed is a process for the catalytic reduction of a nitro group-containing pollutant or waste compound, comprising:
a) obtaining an environmental or waste product having a nitro group containing compound at a concentration of less than about 50 mM;
b) adding a bisulfite compound to the environmental or waste product in an amount from about 2.0 to about 8.0 molar equivalents to the nitro group of the nitro group-containing compound;
c) mixing an aqueous solution of a catalyst with the environmental or waste product, wherein the volume of the aqueous solution is from about 2% to about 20% of the volume of the sample, and wherein the catalyst is present in an amount from about 0.05 to about 1 molar equivalents to the nitro group of the nitro group-containing compound; and
d) reacting the nitro group-containing compound with the bisulfite compound in the presence of the catalyst under reaction conditions of temperature in the range from about 20° C. to about 70° C. and atmospheric pressure for a period from about 10 minutes to about 72 hours to obtain the amine product.

In an embodiment, the nitro group-containing pollutant or waste compound in the environmental or waste product is reduced to the amine product for environmental remediation.

In an embodiment, the environmental remediation process further comprises adding a solvent to the environmental or waste product, and in some embodiments the solvent is dimethylsulfoxide (DMSO).

In an embodiment, disclosed is a process for the catalytic reduction of a nitro group-containing compound to an amine product, comprising:
a) dissolving the nitro group-containing compound in a solvent;
b) reacting the nitro group-containing compound with a bisulfite compound in the presence of a catalyst; and
c) reducing the nitro group-containing compound to the amine product.

In further embodiments, steps (a) and (b) are performed simultaneously or consecutively.

In further embodiments, the nitro group-containing compound is selected from the group consisting of aliphatic, aromatic and heterocyclic compounds and mixtures thereof.

In further embodiments, the catalyst is a transition metal catalyst containing an ion selected from the group consisting of manganese, iron, cobalt, nickel, and copper, and including salts, complexes, and mixtures thereof.

In further embodiments, the bisulfite compound is selected from the group consisting of sodium bisulfite, potassium bisulfite, and ammonium bisulfite, and mixtures thereof.

In further embodiments, the bisulfite compound is sodium bisulfite.

In further embodiments, about 95% of the nitro-group starting material is reduced to an amine product.

In further embodiments, the reaction is conducted under atmospheric pressure.

In further embodiments, the nitro group of the nitro group-containing compound is selectively reduced by the bisulfite reactions without side reactions with one or more substituents on the nitro group-containing compound.

In further embodiments, the process further comprises adding a buffering agent to the process, wherein the buffering agent is a tertiary organic amine selected from triethylamine (TEA), triethanolamine(TEOA), N, N-diethylisopropylamine, N, N-diisopropylethylamine (DIPEA), tripropylamine, trioctylamine or basic inorganic salts selected from sodium acetate, disodium phosphate, sodium sulfite, and sodium hydrosulfite, and mixtures thereof.

In further embodiments, the solvent is dimethylsulfoxide (DMSO).

In further embodiments, the reaction is performed at a temperature between about 20° C. to about 70° C.

In further embodiments, the process further comprises the following steps:
d) monitoring the conversion of the nitro-group containing compound to products by liquid or gas chromatography; and
e) adding a buffering agent to the solvent to push the reaction to completion if the nitro-group containing compound is detected in step (d).

In further embodiments, the bisulfite is formed in situ from a bisulfite precursor.

In an embodiment, disclosed is a process for the catalytic reduction of a nitro group-containing compound to an amine product, comprising:
a) dissolving the nitro-group containing compound in dimethyl sulfoxide (DMSO) solvent at a concentration from about 50 mM to about 1 M;
b) adding sodium bisulfite to the solvent in an amount from about 2.0 to about 8.0 molar equivalents to the nitro group of the nitro group-containing compound;
c) mixing an aqueous solution of a copper catalyst with the solvent, wherein the volume of the aqueous solution is from about 2% to about 20% of the volume of the solvent, and wherein the copper catalyst is present in an amount from about 0.05 to about 1 molar equivalents, or from about 0.05 to about 1 molar equivalents, to the nitro group of the nitro group-containing compound;
d) reacting the nitro group-containing compound with the sodium bisulfite in the presence of the copper catalyst under reaction conditions of temperature in the range from about 20° C. to about 70° C. and atmospheric pressure for a period from about 10 minutes to about 72 hours to obtain the amine product.

In an embodiment, disclosed is an environmental remediation process for the catalytic reduction of a nitro group-containing pollutant or waste compound, comprising:
a) obtaining an environmental or waste product having a nitro group containing compound at a concentration of less than about 50 mM;
b) adding a bisulfite compound to the environmental or waste product in an amount from about 2.0 to about 8.0 molar equivalents to the nitro group of the nitro group-containing compound;
c) mixing a catalyst with the environmental or waste product, wherein the catalyst is present in an amount from about 0.05 to about 1 molar equivalents to the nitro group of the nitro group-containing compound; and
d) reacting the nitro group-containing compound with the bisulfite compound in the presence of the catalyst under reaction conditions of temperature in the range from about 20° C. to about 70° C. and atmospheric pressure for a period from about 10 minutes to about 72 hours to obtain the amine product.

### BRIEF DESCRIPTION OF THE DRAWINGS

FIG. 1 presents a typical HPLC spectrum for monitoring a typical reaction (methyl-4-fluoro-3-nirobenzoate as the example of starting nitro compound);

FIG. 2 presents a typical HPLC weight assay working plot (excellent linear relation at concentration below 0.5 mg/ml for the reduction of methyl-4-fluoro-3-nirobenzoate);

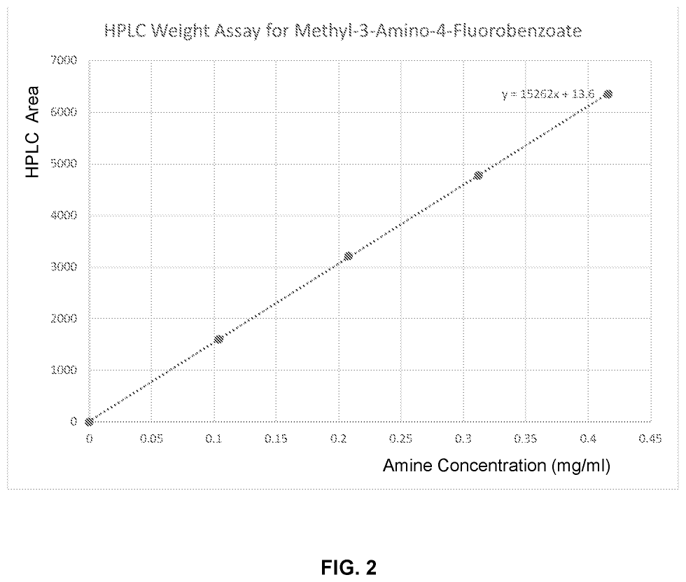

FIG. 3 Presents the reaction without catalyst but with triethylamine at 50° C., after overnight by HPLC too many impurities are formed;

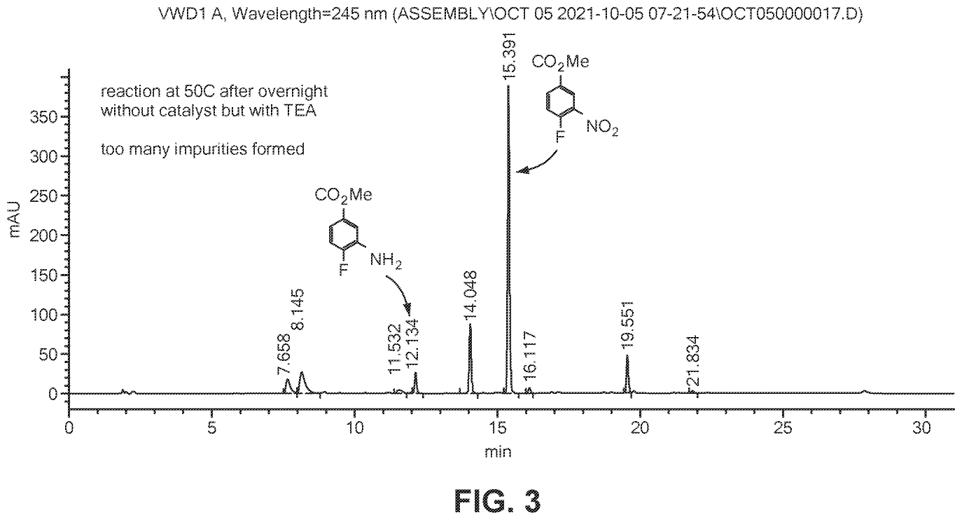

FIG. 4A presents the comparison result using HPLC spectrum for the reaction at room temperature with Na₂S₂O₄ as reductant with catalyst;

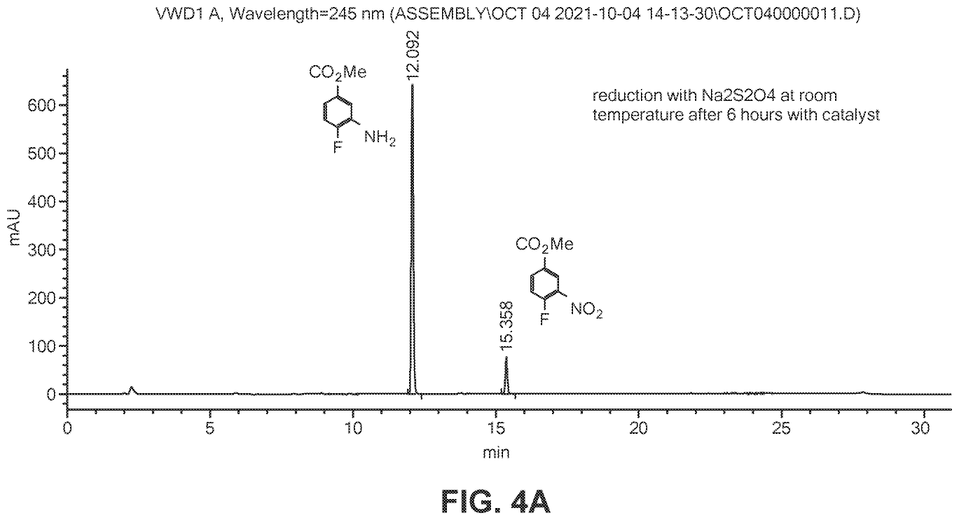

FIG. 4B presents the comparison result using HPLC spectrum for the reaction at room temperature with Na₂S₂O₄ as reductant without catalyst, yielding low conversion and many impurities; and

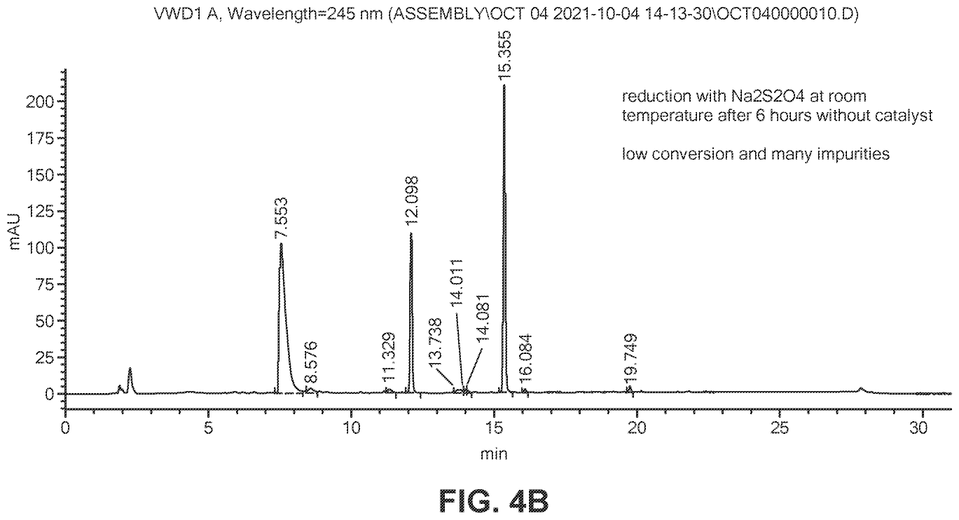

FIG. 4C presents the comparison result using HPLC spectrum for the reaction at 50° C. with Na₂S₂O₄ as reductant without catalyst, yielding a messy reaction.

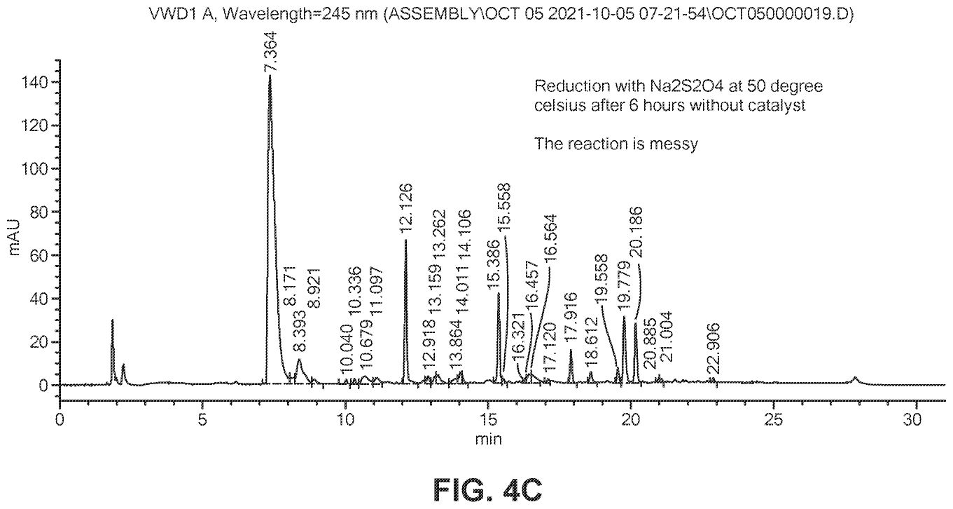

### DETAILED DESCRIPTION

In one embodiment of the present invention, the disclosed methods apply to various different nitro compounds, including aliphatic, aromatic and heterocyclic compounds.

As used herein, the term “aliphatic compound” means an organic compound containing straight, branched and cyclic carbon chains, in which single carbon-carbon bonds (alkanes), double carbon-carbon bonds (alkenes) or triple carbon-carbon bonds (alkynes) may be present. In general, an “aliphatic compound” typically contains one or more spa hybridized carbon atoms. In some embodiments, an “aliphatic compound” may contain one or more heteroatoms such as N, O, S, Si, and B, among others known to persons of skill in the art. An aliphatic compound may be inclusive of cycloalkyl rings and/or fused cycloalkyl rings, optionally containing one or more double or triple bonds.

As used herein, the term “alkyl” refers to a group having carbon-carbon single bonds, “alkenyl” refers to a group having one or more carbon-carbon double bonds, and “alkynyl” refers to a group having one or more carbon-carbon triple bonds. Alkyl, alkenyl, and alkynyl groups may be defined by a number of total carbon atoms, such as C₂-C₁₄, and the double or triple bond(s), if any, can be at any position. Alkyl, alkenyl, and alkynyl groups may be substituted with one or more heteroatoms such as N, O, and S.

The term “aromatic compound” means an organic compound having one or more carbon rings with aromatic structure. The aromatic compounds can be monocyclic or polycyclic. Examples of aromatic compounds are benzene, naphthalene, anthracene and phenanthrene. In general, an “aromatic compound” typically contains one or more sp² hybridized carbon atoms. In some embodiments, an “aromatic compound” may contain one or more heteroatoms such as N, O, and S, among others known to a person of skill in the art.

The term “heterocyclic compound” means an aromatic organic compound as defined above in which one or more carbons of an aromatic ring are replaced by oxygen, sulphur or nitrogen atoms. Examples of heterocyclic compounds are pyridine, pyrimidine, pyrazine, pyridazine, triazine, furan, thiophene, pyrrole, imidazole, pyrazole, thiazole, isothiazole, oxazole, isoxazole, triazole, tetrazole, quinoline, isoquinoline, indole, benzofuran, benzothiophene, benzothiazole, indazole, benzoimidazole, carbazole, 1,2,4-thiadiazole and the like.

The term “quantitative conversion” as used herein means that most or all nitro group starting material is reduced to an amine product. Typically, “quantitative conversion” means that at least about 95% of nitro group starting material is reduced to an amine product. The conversion may be monitored by HPLC detection of the starting material. In an alternative embodiment, at least about 96%, or at least about 97%, or at least about 98%, or at least about 99%, or 100% conversion is achieved.

Many non-toxic late transition metal ions (non-noble) are useful as catalysts for the present invention, such as manganese, iron, cobalt, nickel, and copper. For cost reasons, iron and copper are preferred. Based on the solubility of these ion salts, copper salt is most preferred. In an embodiment, the catalyst species is MnCl₂, FeCl₂, CoCl₂, Ni(OAc)₂, or CuSO₄. In an embodiment, the copper salt is any Cu(II) salt. In another embodiment, the copper salt is, Cu(OAc)₂, CuSO₄, CuCl₂. In another embodiment, the copper salt is CuSO₄. In another embodiment, the copper salt is Cu(OAc)₂.

Bisulfites are common inorganic salts of bisulfite and the materials that can be converted into inorganic salts of bisulfite under dissolution or reaction conditions. Inorganic salts can be alkali metals, alkaline earth metals, and ammonium, as long as they have reasonable solubility in reaction media. In some embodiments, the bisulfite may be sodium hydrosulfite (Na₂S₂O₄), potassium hydrosulfite (K₂S₂O₄), sodium sulfite (Na₂SO₃), and potassium sulfite (K₂SO₃), and/or mixtures thereof. In further embodiments, the bisulfite may be ammonium bisulfite (NH₄HSO₃). In a particular embodiment, the bisulfite is sodium bisulfite. It can be appreciated that, due to their nature, the chemical formulas provided are approximate and non-limiting. Generally, the term “sodium bisulfite” means any mixture of salts that dissolves in water to yield sodium and bisulfite ions. The same holds true for any other salt such as potassium or ammonium salts of bisulfite. Further embodiments are inclusive of additional salts, such as rubidium, cesium, calcium, magnesium, strontium, and mixtures thereof, including mixtures with potassium, sodium, and/or ammonium.

Bisulfites may take on various forms for a given counterion, such as sodium. For instance, sodium bisulfite may include sodium hydrosulfite (Na₂S₂O₄), sodium bisulfite (NaHSO₃), sodium sulfite (Na₂SO₃), and sodium metabisulfite (Na₂S₂O₅), and any mixtures thereof. These exemplary sodium bisulfites are shown below in Table 1, and it can be appreciated that sodium may be substituted for any other appropriate ion as would be understood by a person of skill in the art, such as potassium or ammonium as non-limiting examples.

TABLE 1
Exemplary sodium bisulfites

| Name | Formula | Exemplary Structure |
|---|---|---|
| sodium bisulfite | NaHSO₃ | 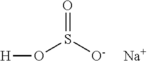 |
| sodium hydrosulfite | Na₂S₂O₄ | 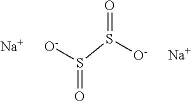 |
| sodium sulfite | Na₂SO₃ | 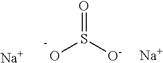 |
| sodium metabisulfite | Na₂S₂O₅ | 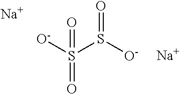 |

Generally, any bisulfite or material forming bisulfite in situ is contemplated. Bisulfite, as it exists under reaction conditions, is generally described by the below formula representation of its two tautomers:

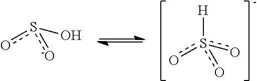

Bisulfite and bisulfite-forming materials are contemplated to include any material which yields bisulfite ions upon dissolution in a suitable solvent, including aqueous and non-aqueous solvents. Bisulfites and bisulfite-forming materials may contain water in their isolated or crystal forms. It is also contemplated that sulfite compounds may be used in place of bisulfite compounds, as sulfite (SO₃²⁻) is the conjugate base of bisulfite (HSO₃⁻).

Any suitable solvent is contemplated for use in the disclosed methods, such as those disclosed in the CRC Handbook of Chemistry and Physics, 102^{nd} edition, 2021. Preferably, the solvent is chosen to give quantitative conversion. In some embodiments, DMSO is preferably the solvent. In certain embodiments, DMSO may comprise one or more co-solvents, or may have one or more co-solvents added to it during the disclosed processes. In certain embodiments, the solvent is ≥99.0, ≥99.5, or ≥99.9% pure DMSO excluding co-solvent, if any. In further embodiments, the solvent may be a sulfoxide or a polar solvent. In further embodiments, the solvent may be an ionic liquid.

In some embodiments, water is used as the co-solvent. The water content in such co-solvent is preferred in the range from about 2 vol % to about 20 vol % with DMSO. In some embodiments, the range may be from about 5% to about 18%, or about 7% to about 15%, or about 10% to about 14%.

In alternative embodiments, a non-aqueous solvent is the co-solvent. The non-aqueous co-solvent content in the solvent is preferred in the range from about 2 vol % to about 20 vol % with DMSO. In some embodiments, the range may be from about 5% to about 18%, or about 7% to about 15%, or about 10% to about 14%. Generally, the non-aqueous co-solvent is a polar solvent.

In the one embodiment of the present invention, the preferred temperature ranges from about 20° C. to about 70° C. The reaction rate is faster for aromatic nitro compounds with electron-withdrawing groups than the aromatic nitro compounds with electron-donating groups. For example, Methyl-4-fluoro-3-nitrobenzoate gave quantitative conversion in 20 hours at 25° C., but there was almost no reaction for 3,4-dimethoxy nitrobenzene after 20 hours at 25° C. Impurities may be formed at reaction temperatures higher than about 70° C.

In yet another embodiment of the present invention, one or more strong acidic protons may be generated as the reaction progresses, and buffering materials can be added into the reaction mixture to push the reaction to completion. All basic materials with aqueous solution in the range of about pH4.0 to about pH11 can be used as buffering materials, including but not limited to tertiary organic amines and inorganic salts. Tertiary organic amines include all such as triethylamine (TEA), N, N-diethylisopropylamine, N, N-diisopropylethylamine (DIPEA), tripropylamine, trioctylamine, etc. TEA is preferred. Common inorganic salts such as disodium phosphate, sodium sulfite, sodium hydrosulfite (sodium dithionite), organic salts such as sodium acetate, are also preferred. In another embodiment, basic materials with aqueous solution in the range of about pH 3.0 to pH 12.0 are used as buffering materials.

In still another embodiment of the present invention, a buffering material to nitro group molar ratio may range from about 1 to about 3, depending on the property of the buffering material. For example, one equivalent of sodium hydrosulfite, two equivalent of TEA, sodium sulfite or disodium phosphate are enough to push reaction to completion; three equivalent of sodium acetate may be employed for quantitative conversion.

The reactions herein usually complete in a time ranging from about 2 to about 72 hours, typically within about 24 hours. The present reactions also generally exhibit high chemo-selectivity which allows the nitro groups to be reduced, even in the presence of much other potentially reducible functionality, which are in many cases left unchanged. In one embodiment, the nitro derivative that undergoes the reduction to amine also contains at least one functional group selected from the group consisting of a double or triple carbon-carbon bond; halogen; C₁-C₄ hydroxyalkyl, preferably hydroxymethyl; allyl ether; alkyl ether; C₁-C₄ acylamino; nitrile; carboxyl; carboxyl or thio-carboxyl ester selected from C₁-C₄ alkyl ester, C₆-C₁₄ aryl ester or C₇-C₁₈ aryl alkyl ester, preferably benzyl ester; sulfoxide or sulfone groups.

In an embodiment, disclosed is a process for the catalytic reduction of the nitro group of a nitro-group containing aromatic compound. In an embodiment, disclosed is a process for preparing amino compounds of formula

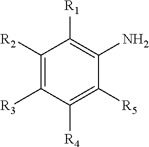

comprising:
a) dissolving a nitro group-containing compound according to formula I in a solvent:

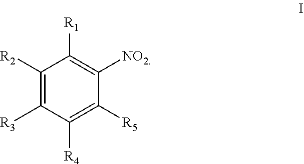

wherein R₁, R₂, R₃, R₄, and R₅ are each independently H, C₁-C₁₄ alkyl, C₁-C₁₄ alkenyl, C₁-C₁₄ alkynyl, halogen, C₁-C₄ hydroxyalkyl, hydroxymethyl, allyl ether, alkyl ether, C₁-C₄ acylamino, nitrile, hydroxy, carboxyl, C₁-C₄ alkyl carboxyl or thio-carboxyl ester, C₆-C₁₄ aryl carboxyl or thio-carboxyl ester, C₇-C₁₈ aryl alkyl carboxyl or thio-carboxyl ester, benzyl ester; sulfoxide, sulfone group, nitro group, amine, methyl, ethyl, propyl, butyl, methoxy, ethoxy, propoxy, isopropoxy, butoxy, NR₆, OR₆, C═OR₆, C═OOR₆, NC═OR₆, or C═ONR₆ optionally substituted at one or more positions with an alkyl, alkenyl, alkynyl, halogen, nitrile, carboxyl or thio-carboxyl ester, R₆, NR₆, OR₆, C═OR₆, C═OOR₆, NC═OR₆, or C═ONR₆;
wherein any two adjacent R₁, R₂, R₃, R₄, and R₅ form a portion of a fused three, four, five, six, seven, or eight-membered cycloalkyl or aromatic ring, or a fused bicyclic or tricyclic cycloalkyl or aromatic ring, optionally having 0, 1, 2, or 3 heteroatoms selected from N, S, and O, and optionally substituted with one or more of R₆, NR₆, OR₆, C═OR₆, C═OOR₆, NC═OR₆, or C═ONR₆;
wherein R₆ is H, halogen, hydroxy, carbonyl, methyl, ethyl, propyl, butyl, methoxy, ethoxy, propoxy, isopropoxy, butoxy, C₁-C₁₄ alkyl, C₁-C₁₄ alkenyl, C₁-C₁₄ alkynyl, aryl, benzyl, C₁-C₄ hydroxyalkyl, hydroxymethyl, allyl ether, alkyl ether, C₁-C₄ acylamino, nitrile, carboxyl, C₁-C₄ alkyl carboxyl or thio-carboxyl ester, C₆-C₁₄ aryl carboxyl or thio-carboxyl ester, C₇-C₁₈ aryl alkyl carboxyl or thio-carboxyl ester, benzyl ester; sulfoxide, and sulfone group.
b) reacting the nitro group-containing compound with a bisulfite compound in the presence of a catalyst;
c) optionally, adding to the solvent a buffering agent comprising tertiary organic amines or basic inorganic salts; and
d) reducing the nitro group-containing compound to the amine product.

In an embodiment, disclosed is a process for the catalytic reduction of the nitro group of a nitro-group containing aliphatic compound. In an embodiment, disclosed is a process for preparing amino compounds of formula

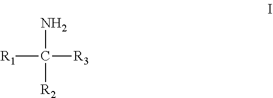

comprising:
a) dissolving a nitro group-containing compound according to formula II in a solvent:

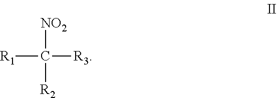

wherein the nitro-containing aliphatic compounds is an alkyl, alkenyl, alkynyl, cycloalkyl, or fused cycloalkyl comprising one or more nitro groups at any position, and
wherein R₁, R₂, and R₃ are each independently selected from the group consisting of one or more of H, C₁-C₁₄ alkyl, C₁-C₁₄ alkenyl, C₁-C₁₄ alkynyl, halogen, hydroxy, C₁-C₄ hydroxyalkyl, hydroxymethyl, allyl ether, alkyl ether, C₁-C₄ acylamino, nitrile, carboxyl, C₁-C₄ alkyl carboxyl or thio-carboxyl ester, C₆-C₁₄ aryl carboxyl or thio-carboxyl ester, C₇-C₁₈ aryl alkyl carboxyl or thio-carboxyl ester, benzyl ester; sulfoxide, sulfone group, nitro group, amine, methyl, ethyl, propyl, butyl, methoxy, ethoxy, propoxy, isopropoxy, butoxy, NR₄, OR₄, C═OR₄, C═OOR₄, NC═OR₄, or C═ONR₄ optionally substituted at one or more positions with an alkyl, alkenyl, alkynyl, halogen, nitrile, carboxyl or thio-carboxyl ester, R₄, NR₄, OR₄, C═OR₄, C═OOR₄, NC═OR₄, or C═ONR₄;
wherein any two of R₁, R₂, or R₃ optionally form a cycloalkyl, optionally fused to further cycloalkyl or aromatic rings; and
wherein R₄ is H, halogen, hydroxy, carbonyl, methyl, ethyl, propyl, butyl, methoxy, ethoxy, propoxy, isopropoxy, butoxy, C₁-C₁₄ alkyl, C₁-C₁₄ alkenyl, C₁-C₁₄ alkynyl, aryl, benzyl, C₁-C₄ hydroxyalkyl, hydroxym ethyl, allyl ether, alkyl ether, C₁-C₄ acylamino, nitrile, carboxyl, C₁-C₄ alkyl carboxyl or thio-carboxyl ester, C₆-C₁₄ aryl carboxyl or thio-carboxyl ester, C₇-C₁₈ aryl alkyl carboxyl or thio-carboxyl ester, benzyl ester; sulfoxide, and sulfone group.
b) reacting the nitro group-containing compound with a bisulfite compound in the presence of a catalyst; and
c) reducing the nitro group-containing compound to the amine product.

In an embodiment, the nitro group of the aromatic compound is aryl. In an embodiment, the nitro group of the aromatic compound is at a position other than aryl. In an embodiment, the aromatic or aliphatic nitro-group containing compound comprises two or more nitro groups.

The processes of the present disclosure may be carried out in various permutations of steps without departing from the scope of the disclosure. Some exemplary steps follow and may be carried out in any order so long as the conversion of nitro group to amine is achieved. It can be appreciated that a person of skill in the art would be able to monitor the conversion by any known useful techniques, including chromatography and not limited to gas chromatography (GC) or liquid chromatograph (LC), including high-pressure liquid chromatography (HPLC).

In an embodiment, the process is initiated by dissolving the nitro group-containing compound in a non-aqueous solvent, followed by adding bisulfite to the solvent. Alternatively, the bisulfate and nitro group-containing compound may be mixed concurrently in the solvent for dissolution. Typically, an aqueous solution of the catalyst is then added to the solvent. In some embodiments, the catalyst may be dissolved in the non-aqueous solvent first, or after adding a water or polar co-solvent to the non-aqueous solvent, depending upon its solubility. The goal of these steps is to prepare a solution of the nitro-group containing compound, the bisulfite, and the catalyst and any steps to yield this result are contemplated. In exemplary embodiments, the non-aqueous solvent is DMSO.

The reaction may proceed to an extent once the nitro-group containing compound, the bisulfite, and the catalyst are solubilized in the non-aqueous solvent and water or polar co-solvent. The reaction may then be monitored via chromatography, such as HPLC, to detect the nitro-group starting material. Alternatively, the product(s) may be detected. The goal of the monitoring step is to determine whether or not a buffering agent needs to be added to push the reaction to completion. Typically, a buffering agent is added in an amount of at least about 0.5 molar equivalents to the nitro group. In some embodiments, at least about 0.5, or about 1.0, or about 1.5, or about 2.0, or about 2.5, or about 3.0, or about 3.5, or about 4.0, or about 4.5, or about 5.0, or about 6.0, or about 7.0, or about 8.0, or about 9.0, or about 10.0 molar equivalents of the buffering agent are added. In an embodiment, the amount of buffering agent may be determined by allowing the initial reaction without buffering agent to proceed to an intermediate endpoint by monitoring the conversion. The intermediate endpoint, which may be an estimated conversion percentage, may be used to infer the amount of buffering agent needed. Continuous monitoring is contemplated.

One or more monitoring steps may be performed depending upon the reaction progress. Alternatively, in some embodiments, it may be preferable to perform repeated or slow, continuous additions of the buffering agent. It can be appreciated that any method for adding the buffering agent is contemplated. In alternative embodiments, the monitoring step may be eliminated, and the buffering agent may be added at a pre-determined or estimated amount without subsequent monitoring.

Additional bisulfite may also be added throughout the course of the reaction as needed. The additional bisulfite, if needed, may be added before, after, or concurrently with any additions of buffering agent. It can be appreciated that a person of skill in the art can determine whether or not additional bisulfite and/or buffering agent is needed to push the reaction to completion.

The reaction is typically carried out until quantitative conversion is achieved. The reaction time may depend upon the particular substrate and buffering agent addition, concentration, temperature, or other factors. In some embodiments, the concentration of the nitro-containing compound is about 100 mmol or less in the non-aqueous solvent, such as DMSO. In some embodiments, the concentration is above 100 mmol if the reaction proceeds to yield a clean product. In some embodiments, the concentration is above 1 M, or about 1M, or about 800 mM, or about 600 mM, or about 500 mM, or about 400 mM, or about 300 mM, or about 200 mM, or about 100 mM, or about 80 mM, or about 60 mM, or about 50 mM, or about 40 mM, or about 30 mM, or about 20 mM, or about 10 mM, or about 1 mM, or about 0.5mM, or less.

In some embodiments, the disclosed process is useful for environmental remediation. In some embodiments, the process is utilized on an environmental or waste product containing one or more nitro group-containing compounds. The concentration of the nitro group-containing compounds is typically lower (i.e., lower than about 50 mM) in such environmental or waste products compared to synthetic methodologies (i.e., higher than about 50 mM), however the present processes are not limited by these illustrative concentration values. The environmental or waste product may be an aqueous liquid, a non-aqueous liquid, or a solid and a person of skill in the art would appreciate that a solvent as disclosed herein could be incorporated with the environmental or waste product in order to perform the disclosed processes. In general, “environmental remediation” in the context of environmental or waste products containing one or more nitro group-containing compounds means partial or complete reduction of the nitro group-containing compounds to amine products.

The amount of bisulfite added is typically relative to the amount of nitro-containing compound. In some embodiments, the molar ratio of bisulfite to nitro group is about 4.0 or higher. In some embodiments, a molar ratio less than 4.0 is tolerated, as determinable by a person of skill in the art. In some embodiments, the molar ratio is about 2.0, or about 3.0, or about 4.0, or about 5.0, or about 6.0, or about 7.0, or about 8.0, or about 9.0, or about 10.0.

The amount of catalyst to be added to the reaction may vary in amount depending upon the particular reaction. Typically, the concentration of the catalyst is from about 0.5 mM to about 100 mM. With respect to the molar amount of nitro group, about 0.01, or about 0.05, or about 0.1, or about 0.2, or about 0.3, or about 0.4, or about 0.5, or about 0.6, or about 0.7, or about 0.8, or about 0.9, or about 1.0, or about 1.5, or about 2.0, or about 2.5, or about 3.0 or about 4.0 or about 5.0 molar equivalents of catalyst may be added or included.

The amount of buffering agent added to reach quantitative conversion may vary according to the substrate scope and other reaction conditions. In some embodiments, the amount of buffering agent added is about 0.5, or about 1.0, or about 1.5, or about 2.0, or about 2.5, or about 3.0, or about 4.0, or about 5.0, or about 6.0, or about 7.0, or about 8.0, or about 10.0 molar equivalents with respect to the nitro group. The amount added may depend upon the reaction progress, or upon the basicity of the particular buffering agent added. Typically, a more strongly basic buffering agent will be added in a smaller molar equivalent while a weakly basic buffering agent will be added in a larger molar equivalent for otherwise identical reactions.

The processes disclosed herein may be effective under economical reaction conditions. Unlike known systems, which often require drastic conditions and heating of the reaction mixture, the mild reaction conditions, and operational simplicity of the present process make it attractive for industrial use.

### EXAMPLES

It is appreciated that certain features of the disclosure, which are, for clarity, described in the context of separate exemplary embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments unless the embodiment is inoperative without those elements. It will be apparent to those skilled in the art that many modifications, both to materials and methods, may be practiced without departing from the scope of the disclosure.

Various embodiments and aspects of the present invention as delineated hereinabove are shown in the following experimental examples.

### Example 1

### Reduction of Aliphatic Nitro Compound

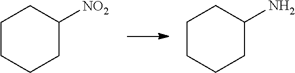 [SMILES: O=[N+]([O-])C1CCCCC1 ; NC1CCCCC1]

The mixture of nitrocyclohexane (3.0 mmol) and sodium bisulfite (12 mmol, 4.0 eq) in 15 ml of DMSO was stirred under nitrogen at room temperature for about 30 minutes. The solution of copper sulfate (0.06 mmol, 2.0 mol %) in water (0.5 ml) was added to the reaction mixture at room temperature with stirring under nitrogen. The reaction mixture was stirred at room temperature for NLT (not less than) one hour, then heated up to 50° C.

The reaction was monitored by HPLC, TEA (2.0 eq) and extra 4.0 eq of sodium bisulfite were added portion-wise corresponding to conversion. For example, after the conversion was 30%, 30% of TEA (2.0 eq) and 30% of the extra 4.0 eq of sodium bisulfite (1.2 eq of sodium bisulfite) were added. The reaction mixture was analyzed by HPLC weight assay with authentic cyclohexylamine. The yield was quantitative (99.6%) by weight assay.

For comparison, a reaction with 80 mmol (10.4 g) of nitrocyclohexane was carried out following similar procedure for small scale reaction (3.0 mmol reaction). 100 ml of DMSO, 4 ml of water, 0.35 g of CuSO₄5H₂O, 0.64 mole of sodium bisulfite and 0.16 mole of triethylamine were used. After the reaction reached more than 99% conversion (99.6% conversion), the reaction was quenched with cold water (50 ml) at temperature <10° C. The pH of the quenched reaction mixture was adjusted with saturated trisodium phosphate water solution to pH7. The HPLC weight assay gave 99.5% product yield. For product isolation: The most of inorganic salts were filtered out at temperature about 3° C. The inorganic salt was washed with diethyl ether to collect product absorbed by solid. The product was isolated via vacuum distillation (63-66° C./80 torr). 93.2% of cyclohexylamine (7.40 g) was obtained as pure product and 6.3% product left in residue. This result confirmed that HPLC weight assay is a reliable method for calculating product yield. The product was characterized by its HPLC retention time and NMR. ¹H-NMR (CDCl₃): (Chemical Formula: C₆H₁₃N, Molecular Weight: 99.18): 2.53 (m, 1H), 1.65 (m, 5H), 1.13 (m, 7H). ¹³C-NMR (CDCl₃): 50.42, 36.88, 25.64, 25.10.

The reaction was also carried out at 3.0 mmol of nitro cyclohexane with sodium hydrosulfite (Na₂S₂O₄, 7.5 mmol, 2.5 eq) at room temperature (no warmup needed). TEA and extra sodium hydrosulfite were added portion-wise corresponding the reaction conversion. 2.0 eq of TEA and extra 2.0 eq of sodium hydrosulfite were needed to reach reaction completion.

### Example 2

The same procedure as described in Example 1 was used to reduce functionalized aliphatic substrates, such as 2-nitropropanol, obtaining the corresponding amines in a quantitative yield.

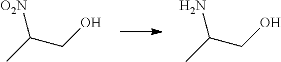

### Example 3

### Reduction of Aromatic Nitro Derivatives with Halide Functional Group

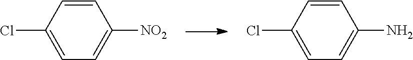 [SMILES: O=[N+]([O-])c1ccc(Cl)cc1 ; Nc1ccc(Cl)cc1]

The same procedure as described in Example 1 was used. At 50° C. reaction temperature, 4.0 eq of NaHSO₃ and 2.0 eq of TEA were needed to reach reaction completion.

### Example 4

Reduction of Aromatic Dinitro Derivatives to Di-Amine Product

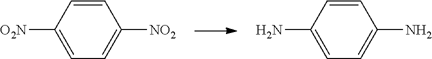 [SMILES: O=[N+]([O-])c1ccc([N+](=O)[O-])cc1 ; Nc1ccc(N)cc1]

The same procedure as described in Example 1 was used. At 50° C. reaction temperature, 9.0 eq of NaHSO₃ and 4.0 eq of TEA were needed to reach reaction completion.

### Example 5

### Reduction of Aromatic Dinitro Derivatives to Di-Amine Product

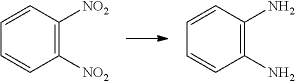 [SMILES: O=[N+]([O-])c1ccccc1[N+](=O)[O-] ; Nc1ccccc1N]

The same procedure as described in Example 1 was used. At 50° C. reaction temperature, 8.0 eq of NaHSO₃ and 4.0 eq of TEA were needed to reach reaction completion.

### Example 6

### Reduction of Aromatic Dinitro Derivatives to Monoamine Product

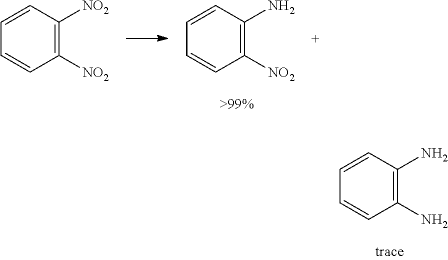 [SMILES: O=[N+]([O-])c1ccccc1[N+](=O)[O-] ; Nc1ccccc1[N+](=O)[O-] ; Nc1ccccc1N]

The same procedure as described in Example 1 was used. At 50° C. reaction temperature, 4.0 eq of NaHSO₃ and 2.0 eq of TEA were needed to reach reaction completion.

### Example 7

### Reduction of Nitrobenzene

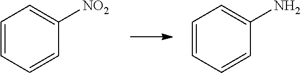 [SMILES: O=[N+]([O-])c1ccccc1 ; Nc1ccccc1] [0141] A) The same procedure as described in Example 1 was used. At 50° C. reaction temperature, 5.0 eq of NaHSO₃ and 2.0 eq of TEA were needed to reach reaction completion.
B) The same procedure as described in Example 3 was used. At 50° C. reaction temperature, 2.5 eq of Na₂S₂O₄ and 1.0 eq of TEA were needed to reach reaction completion.

### Example 8

### Reduction of Para-Nitroaniline

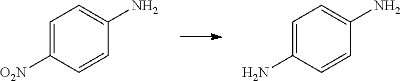 [SMILES: Nc1ccc([N+](=O)[O-])cc1 ; Nc1ccc(N)cc1] [0144] A) The same procedure as described in Example 1 was used. At 50° C. reaction temperature, 6.0 eq of NaHSO₃ and 2.0 eq of TEA were needed to reach reaction completion.
B) The same procedure as described in Example 3 was used. 50° C. reaction temperature, 3.5 eq of Na₂S₂O₄ and 1.0 eq of TEA were needed to reach reaction completion.

### Example 9

### Reduction of Meta-Nitroaniline

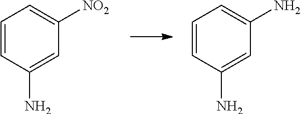 [SMILES: Nc1cccc([N+](=O)[O-])c1 ; Nc1cccc(N)c1] [0147] A) The same procedure as described in Example 1 was used. At 50° C. reaction temperature, 6.0 eq of NaHSO₃ and 2.0 eq of TEA were needed to reach reaction completion.
B) The same procedure as described in Example 3 was used. At 50° C. reaction temperature, 2.5 eq of Na₂S₂O₄ and 1.0 eq of TEA were needed to reach reaction completion.

### Example 10

### Reduction of 4-Nitrobenzoic Acid

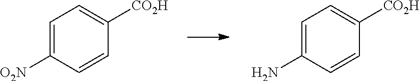 [SMILES: O=C(O)c1ccc([N+](=O)[O-])cc1 ; Nc1ccc(C(=O)O)cc1] [0150] A) The same procedure as described in Example 1 was used. At 50° C. reaction temperature, 5.0 eq of NaHSO₃ and 2.0 eq of TEA were needed to reach reaction completion. The addition of TEA should be very slow, otherwise, trace to about 5% of amide product will be formed.
B) The same procedure as described in Example 3 was used. At 50° C. reaction temperature, 3.0 eq of Na₂S₂O₄ and 1.0 eq of TEA were needed to reach reaction completion. The addition of TEA should be very slow, otherwise, trace to about 8% of amide product will be formed.

### Example 11

### Reduction of 3-Nitrobenzoic Acid

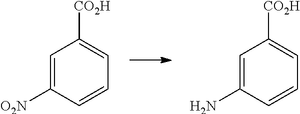 [SMILES: O=C(O)c1cccc([N+](=O)[O-])c1 ; Nc1cccc(C(=O)O)c1] [0153] A) The same procedure as described in Example 1 was used. At 50° C. reaction temperature, 5.0 eq of NaHSO₃ and 2.0 eq of TEA were needed to reach reaction completion. The addition of TEA should be very slow, otherwise, trace to about 3% of amide product will be formed.
B) The same procedure as described in Example 3 was used. At 50° C. reaction temperature, 3.0 eq of Na₂S₂O₄ and 1.0 eq of TEA were needed to reach reaction completion. The addition of TEA should be very slow, otherwise, trace to about 5% of amide product will be formed.

### Example 12

### Reduction of Nitro Compound with Ether Functional Group

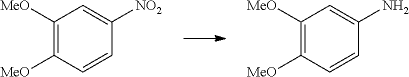 [SMILES: COc1ccc([N+](=O)[O-])cc1OC ; COc1ccc(N)cc1OC] [0156] A) The same procedure as described in Example 1 was used. At 50° C. reaction temperature, 10.0 eq of NaHSO₃ and 2.0 eq of TEA were needed to reach reaction completion.
B) The same procedure as described in Example 3 was used. At 50° C. reaction temperature, 4.5 eq of Na₂S₂O₄ and 1.0 eq of TEA were needed to reach reaction completion.

### Example 13

### Reduction of Nitro Compound with N-Acetyl Functional Group

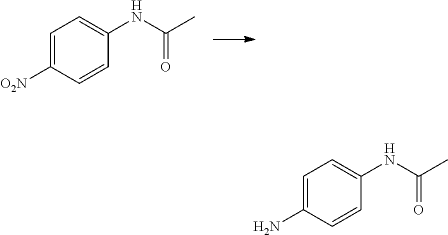 [0159] A) The same procedure as described in Example 1 was used. At 50° C. reaction temperature, 6.0 eq of NaHSO₃ and 2.0 eq of TEA were needed to reach reaction completion.
B) The same procedure as described in Example 3 was used. At 50° C. reaction temperature, 3.5 eq of Na₂S₂O₄ and 1.0 eq of TEA were needed to reach reaction completion.

### Example 14

### Reduction of Heterocyclic Nitro Compound

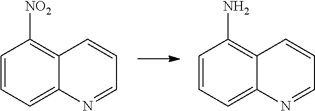 [SMILES: O=[N+]([O-])c1cccc2ncccc12 ; Nc1cccc2ncccc12] [0162] A) The same procedure as described in Example 1 was used. At 50° C. reaction temperature, 4.0 eq of NaHSO₃ and 2.0 eq of sodium hydrosulfite buffer were needed to reach reaction completion.
B) The same procedure as described in Example 3 was used. At 50° C. reaction temperature, 4.0 eq of Na₂S₂O₄ and 1.0 eq of TEA were needed to reach reaction completion.

### Example 15

### Reduction of Nitro Compounds with Nitrile Functional Group

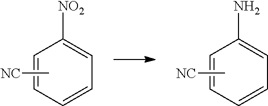

The same procedure as described in Example 1 was used. At 50° C. reaction temperature, 4.0 eq of NaHSO₃ and 2.0 eq of TEA buffer were needed to reach reaction completion. No difference was observed among 2-nitrobenzonitrile, 3-nitrobenzonitrile, and 4-nitrobenzonitrile.

### Example 16

### Reduction of Aromatic Nitro Compound with Other Functional Groups

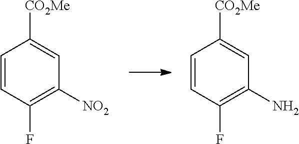 [SMILES: COC(=O)c1ccc(F)c([N+](=O)[O-])c1 ; COC(=O)c1ccc(F)c(N)c1]

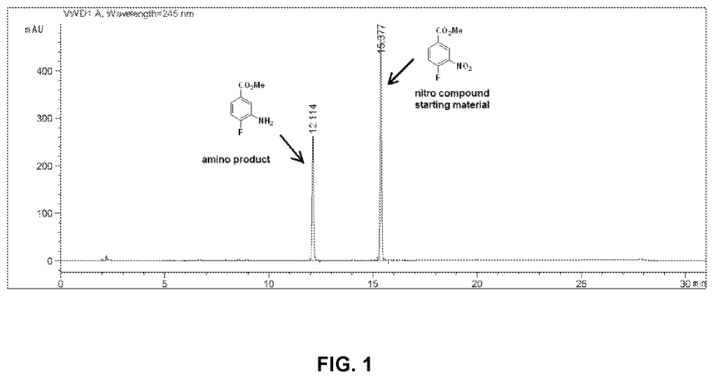

The mixture of Methyl-4-fluoro-3-nitro benzoate (16 g, 80 mmol) and sodium bisulfite (320 mmol, 4.0 eq) in 100 ml of DMSO was stirred under nitrogen at room temperature for about 30 minutes. The solution of copper sulfate (1.6 mmol, 2.0 mol %) in water (4.0 ml) was added to the reaction mixture at room temperature with stirring under nitrogen. The reaction mixture was stirred at room temperature under nitrogen and monitored by HPLC. A typical HPLC spectrum is shown by FIG. 1. HPLC was conducted under the following exemplary conditions:

### Instrument

HPLC with UV detector
Reverse Phase-C18 (4.6 mm×150 mm, 3.5 μm) column
Oven temperature: 30° C.
   Mobile phase A: 0.02% H₃PO₄ in Water: ACN (90:10, v/v), v/v
   For example, accurately transfer 100 mL of ACN and 900 mL of purified water and mix well. Then transfer 0.2 mL of H₃PO₄ and mix well.
   Mobile phase B: 0.02% H₃PO₄ in Water: ACN (10:90, v/v), v/v
   For example, accurately transfer 900 mL of ACN and 100 mL of purified water and mix well. Then transfer 0.2 mL of H₃PO₄ and mix well.

Flow rate: 0.8 ml/min
Injection volume: 5 μL

| | Time (min): | | | | |
|---|---|---|---|---|---|
| | 0 | 20.0 | 25.0 | 25.1 | 31.0 |
| Mobile phase A (%): | 100 | 10 | 10 | 100 | 100 |
| Mobile phase B (%): | 0 | 90 | 90 | 0 | 0 |

TEA (160.0 mmol, 2.0 eq) was added portion-wise corresponding to the conversion. For example, 50% of TEA was added after the conversion reached 50%. Alternatively, TEA could also be added by monitoring the acidity of the reaction mixture without using HPLC. As an example of monitoring the reaction mixture without using HPLC, an aliquoted amount of about 0.5 ml of reaction mixture was added into 10 ml of DI water, and the pH of the aqueous solution was measured. If the pH was pH=1, TEA was added into this aqueous solution to adjust pH≤5.5. The total TEA needed for the reaction mixture was calculated based on the TEA used for the aqueous solution. The reaction mixture was analyzed by HPLC weight assay after completion of the reaction. The yield was 99.7% by weight assay.

The reaction mixture was cooled down to <10° C. after the higher than 99% up to quantitative conversion was obtained (20 hours). Under stirring, two volumes of water (200 ml) was added slowly into the reaction mixture to maintain internal temperature <10° C. during addition. TEA was added dropwise into the quenched reaction mixture to adjust pH7 while maintaining internal temperature <10° C. The resulted reaction mixture was stirred at about 5° C. for NLT 5 hours. Product was isolated via filtration and washed with cold water twice (20 ml×2). 12.4 g (91% isolation yield) of methyl-3-amino-4-fluorobenzoate was obtained after drying under nitrogen flow for 48 hours. The product left in filtrate and cake wash was 1.2 g (8.8%) by weight assay. Methyl-3-amino-4-fluorobenzoate was characterized by comparing it's HPLC retention time and ¹H-NMR with purchased compound. ¹H-NMR in CD₃OD (ppm): 7.5 (m, 1H), 7.3 (m, 1H), 7.0 (m, 1H), 4.8 (s, 2H), 3.8 (s, 3H). The total accountable product was 99.8%. This result confirmed that HPLC weight assay is reliable for calculating product yield.

1. A process for the catalytic reduction of a nitro group-containing compound to an amine product, comprising:
a) dissolving the nitro-group containing compound in a solvent;
b) adding a bisulfite compound to the solvent in an amount from about 2.0 to about 8.0 molar equivalents to the nitro group of the nitro group-containing compound;
c) mixing an aqueous solution of a catalyst with the solvent, wherein the volume of the aqueous solution is from about 2% to about 20% of the volume of the solvent, and wherein the catalyst is present in an amount from about 0.05 to about 1 molar equivalents to the nitro group of the nitro group-containing compound;
d) reacting the nitro group-containing compound with the bisulfite compound in the presence of the catalyst under reaction conditions of temperature in the range from about 20° C. to about 70° C. and atmospheric pressure for a period from about 10 minutes to about 72 hours to obtain the amine product.

2. The process of claim 1, wherein steps, a), b), and c) are performed in any simultaneous or consecutive grouping or ordering

3. The process of claim 1, wherein the nitro group-containing compound is selected from the group consisting of aliphatic, aromatic and heterocyclic compounds and mixtures thereof.

4. The process of claim 1, wherein the catalyst is a transition metal catalyst containing an ion selected from the group consisting of manganese, iron, cobalt, nickel, and copper, and including salts, complexes, and mixtures thereof.

5. The process of claim 1, wherein the bisulfite compound is selected from the group consisting of sodium bisulfite, potassium bisulfite, ammonium bisulfite, and mixtures thereof.

6. The process of claim 1, wherein the bisulfite compound is sodium bisulfite.

7. The process of claim 1, wherein about 95% of the nitro-group starting material is reduced to an amine product.

8. The process of claim 1, wherein the nitro group of the nitro group-containing compound is selectively reduced by the bisulfite reactions without side reactions with one or more sub stituents on the nitro group-containing compound.

9. The process of claim 1, further comprising adding a buffering agent to the process in an amount of from about 1 to about 10 molar equivalents to the nitro group of the nitro group-containing compound, wherein the buffering agent is a tertiary organic amine selected from triethylamine (TEA), triethanolamine(TEOA), N, N-diethylisopropylamine, N, N-diisopropylethylamine (DIPEA), tripropylamine, trioctylamine or basic inorganic salts selected from sodium acetate, disodium phosphate, sodium sulfite, and sodium hydrosulfite, and mixtures thereof.

10. The process of claim 1, wherein the solvent is dimethylsulfoxide (DMSO).

11. The process of claim 8, further comprising the following steps:
e) monitoring the conversion of the nitro-group containing compound to products by liquid or gas chromatography, and
f) adding the buffering agent to the solvent to push the reaction to completion if the nitro-group containing compound is detected in step (d).

12. The process of claim 1, wherein the bisulfite is formed in situ from a bisulfite precursor.

13. A process for the catalytic reduction of a nitro group-containing compound to an amine product, comprising:
a) dissolving the nitro group-containing compound in a solvent;
b) reacting the nitro group-containing compound with a bisulfite compound in the presence of a catalyst; and
c) reducing the nitro group-containing compound to the amine product.

14. The process of claim 13, wherein (a) and (b) are performed simultaneously or consecutively.

15. The process of claim 13, wherein the nitro group-containing compound is selected from the group consisting of aliphatic, aromatic and heterocyclic compounds and mixtures thereof.

16. The process of claim 13, wherein the catalyst is a transition metal catalyst containing an ion selected from the group consisting of manganese, iron, cobalt, nickel, and copper, and including salts, complexes, and mixtures thereof.

17. The process of claim 13, wherein the bisulfite compound is selected from the group consisting of sodium bisulfite, potassium bisulfite, and ammonium bisulfite, and mixtures thereof.

18. The process of claim 13, wherein the bisulfite is sodium bisulfite.

19. The process of claim 13, wherein about 95% of the nitro-group starting material is reduced to an amine product.

20. The process of claim 13, wherein the reaction is conducted under atmospheric pressure.

21. The process of claim 13, wherein the nitro group of the nitro group-containing compound is selectively reduced by the bisulfite reactions without side reactions with one or substituents on the nitro group-containing compound.

22. The process of claim 13, further comprising adding a buffering agent to the process, wherein the buffering agent is a tertiary organic amine selected from triethylamine (TEA), triethanolamine(TEOA), N, N-diethylisopropylamine, N, N-diisopropylethylamine (DIPEA), tripropylamine, trioctylamine or basic inorganic salts selected from sodium acetate, disodium phosphate, sodium sulfite, and sodium hydrosulfite, and mixtures thereof.

23. The process of claim 13, wherein the solvent is dimethylsulfoxide (DMSO).

24. The process of claim 13, wherein the reaction is performed at a temperature between about 20° C. to about 70° C.

25. The process of claim 21, further comprising the following steps:
d) monitoring the conversion of the nitro-group containing compound to products by liquid or gas chromatography, and
e) adding a buffering agent to the solvent to push the reaction to completion if the nitro-group containing compound is detected in step (d).

26. The process of claim 13, wherein the bisulfite is formed in situ from a bisulfite precursor.

27. An environmental remediation process for the catalytic reduction of a nitro group-containing pollutant or waste compound, comprising:
a) obtaining an environmental or waste product having a nitro group containing compound at a concentration of less than about 50 mM;
b) adding a bisulfite compound to the environmental or waste product in an amount from about 2.0 to about 8.0 molar equivalents to the nitro group of the nitro group-containing compound;
c) mixing a catalyst with the environmental or waste product, wherein the catalyst is present in an amount from about 0.05 to about 1 molar equivalents to the nitro group of the nitro group-containing compound; and
d) reacting the nitro group-containing compound with the bisulfite compound in the presence of the catalyst under reaction conditions of temperature in the range from about 20° C. to about 70° C. and atmospheric pressure for a period from about 10 minutes to about 72 hours to obtain the amine product.
